(19) United States
(12) Patent Application Publication
(10) Pub. No.: US 2024/0221911 A1
(43) Pub. Date: Jul. 4, 2024

(54) SYSTEMS AND METHODS FOR REMOTE READING

(71) Applicant: GE Precision Healthcare LLC, Wauwatosa, WI (US)

(72) Inventors: Jessica Wadsworth Bolduc, Craftsbury Common, VT (US); James Conti, Algonquin, IL (US); Vijay Kumar Reddy Arlagadda, Hoffman Estates, IL (US)

(21) Appl. No.: 18/092,050

(22) Filed: Dec. 30, 2022

(51) Int. Cl. G16H 30/40

(52) U.S. Cl. CPC G16H 30/40

(57) ABSTRACT

A system may include a processor-based device storing or accessing a packaging application, wherein the packaging application, when executed by the processor-based device, causes acts such as determining a number of exams is greater than a threshold number based on a schedule and assigning a score to each exam of a plurality of exams based on the schedule and on routing rules to be performed. The packaging application, when executed by the processor-based device, may also create a worklist comprising the plurality of exams, package an exam of the plurality of exams with additional data based on user input, and transmit the package to a third-party reader. The plurality of exams may be ordered in the worklist from a highest score to a lowest score.

### BACKGROUND

The subject matter disclosed herein relates to medical data, and more particularly, to systems and methods to automatically package medical data for third-party readers to decrease turn-around time and increase quality of service provided.

This section is intended to introduce the reader to various aspects of art that may be related to various aspects of the present techniques, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

Non-invasive imaging technologies allow images of the internal structures of a subject (e.g., patient, manufactured good, baggage, package, or passenger) to be obtained non-invasively. In particular, such non-invasive imaging technologies rely on various physical principles, such as the differential transmission of X-rays through the target volume or the reflection of acoustic waves, to acquire data and to construct images or otherwise represent the internal features of the subject. By way of example, in X-ray based imaging technologies, signals representative of an amount or an intensity of radiation may be collected and the signals may then be processed to generate an image that may be displayed for review.

After the image is collected, a physician (e.g., a radiologist) may read the image and make a diagnosis. For example, a worklist may be used to track a status of all received images, such as new images, images in review, images needing review, and images completing review. The radiologist may determine images for review based on the worklist. However, an institution may not have enough radiologists or available radiologists with the right expertise to review the volume of images acquired within the institution in a period of time. As such, turn-around time for images within the institution may increase and quality of service delivered may decrease. Thus, systems and methods to identify and provide images for third-party readers may be beneficial.

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In an embodiment, a system may include a processor-based device storing or accessing a packaging application, wherein the packaging application, when executed by the processor-based device, causes acts such as determining a number of exams is greater than a threshold number based on a schedule and assigning a score to each exam of a plurality of exams based on the schedule and on routing rules to be performed. The packaging application, when executed by the processor-based device, may also create a worklist comprising the plurality of exams, package an exam of the plurality of exams with additional data based on user input, and transmit the package to a third-party reader. The plurality of exams may be ordered in the worklist from a highest score to a lowest score.

In an embodiment, a method may include determining, via a processor, that a number of a plurality of exams is greater than a threshold number based on a schedule of in-house readers, assigning a score to each of the plurality of exams based on routing rules, and creating a worklist comprising the plurality of exams, wherein the plurality of exams is ordered based on the score. The method may also generate, via the processor, a graphical user interface (GUI) comprising the worklist and transmit an exam of the plurality of exams to a third-party reader in response to user input.

In an embodiment, a non-transitory, computer-readable medium comprising computer-readable code, that when executed by one or more processors, causes the one or more processors to perform operations including determining that a number of a plurality of exams is greater than a threshold number based on a schedule of in-house readers, assigning a score to each of the plurality of exams based on routing rules, and creating a worklist comprising the plurality of exams, wherein the plurality of exams is ordered based on their respective scores. The one or more processors may also perform operations including generating a first graphical user interface (GUI) comprising the worklist and the threshold number and receiving a user input selecting an exam from the worklist to transmit to a third-party reader. The one or more processors may then perform operations including identifying one or more priors based on attributes of the exam, compressing the exam and the one or more priors into a package, and transmitting the package to the third-party readers.

Various refinements of the features noted above may exist in relation to various aspects of the present disclosure. Further features may also be incorporated in these various aspects as well. These refinements and additional features may exist individually or in any combination. For instance, various features discussed below in relation to one or more of the illustrated embodiments may be incorporated into any of the above-described aspects of the present disclosure alone or in any combination. The brief summary presented above is intended only to familiarize the reader with certain aspects and contexts of embodiments of the present disclosure without limitation to the claimed subject matter.

### BRIEF DESCRIPTION OF THE DRAWINGS

These and other features, aspects, and advantages of the present invention will become better understood when the following detailed description is read with reference to the accompanying drawings in which like characters represent like parts throughout the drawings, wherein:

FIG. 1 illustrates an embodiment of a work environment including a workstation for acquiring and processing image data, in accordance with aspects of the present disclosure;

FIG. 2 illustrates a flow chart of an example method to predict a need to package exams for third-party readers, in accordance with aspects of the present disclosure;

FIG. 3 illustrates a schematic diagram of a user operating the workstation of FIG. 1 to assign exams for the third-party readers, in accordance with aspects of the present disclosure;

FIG. 4 illustrates a graphical user interface (GUI) displaying attributes of the routing rules, in accordance with aspects of the present disclosure;

FIG. 5 illustrates the GUI of FIG. 4 displaying the routing rules, in accordance with aspects of the present disclosure;

FIG. 6 illustrates a flow chart of an example method for creating a worklist of exams based on the routing rules of FIGS. 4 and 5, in accordance with aspects of the present disclosure;

FIG. 7 illustrates a GUI displaying the worklist of FIG. 6 prior to transmitting the exam to the third-party reader, in accordance with aspects of the present disclosure;

FIG. 8 illustrates a flow chart of an example method to package the exam for the third-party reader, in accordance with aspects of the present disclosure;

FIG. 9 illustrates a GUI displaying the exam and a reference list for packaging for the third-party reader, in accordance with aspects of the present disclosure; and

FIG. 10 illustrates a GUI displaying a status of the exam subsequent to transmitting the package to the third-party reader, in accordance with aspects of the present disclosure.

### DETAILED DESCRIPTION

One or more specific embodiments of the present disclosure are described above. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present invention, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

An institution may implement a picture archiving and communication system (PACS) to manage a lifecycle of digital studies, medical images, and reports. For example, a patient may be scheduled for a magnetic resonance imaging (MRI). The MRI image may be collected and transmitted to the PACS for reading (e.g., reviewing) by a medical professional (e.g., radiologist) within the institution. However, the institution may receive high volumes of studies, images, and reports for reading, reviewing, and making a diagnosis. To manage the items, the PACS may maintain a worklist with the digital studies, medical images, and reports as well as a status (e.g., verified, assigned, reviewed, reported) of the item. The worklist may prioritize certain exams (e.g., emergency, stroke) where turn-around time is important (e.g., priority or emergency reads). Otherwise, the worklist may generally display the exams in a first in, first out order. That is, the worklist may display a first received exam first and the radiologist may review the first received exam first (e.g., first out). After reviewing the item, the radiologist may make a diagnosis and/or a report on the item in the PACS, change the status of the item in the worklist (e.g., from verified to dictated to finalized), and send the diagnosis or report to the referring physician or patient.

In certain instances, the institution may not have enough radiologists to review the digital studies, medical images, and reports in a timely manner. For example, an expected turn-around time for the MRI image may be one week; but due to the high volume of studies and shortage of radiologists, the institution may take two weeks to report on the MRI image. Moreover, the radiologists may have a specialty (e.g., breast, chest, emergency, gastrointestinal, etc.), which limits the type of study, image, or report the radiologist may review. For example, the institution may have three radiologists reading CT scans and only one radiologist reading X-Rays. As such, the turn-around time for X-rays may increase and a quality of service provided may decrease.

Embodiments of the present disclosure are directed to a packaging application that forecasts capabilities of in-house readers and predicts a need for packaging exams (e.g., digital studies, medical images, and reports) for transmittal to third-party reviewers to decrease turn-around time. The packaging application may be integrated with the PACS to create a worklist of exams by monitoring incoming exams, the status of ongoing exams, a number of completed exams, available and scheduled in-house reviewers (e.g., radiologists within the institution) and their bandwidth, and available third-party readers eligible to receive examinations for review. In this manner, the packaging application may determine if the pending exams are likely to be reviewed in a timely manner.

In certain instances, the packaging application may predict a need to package and transmit exams to third-party readers to decrease turn-around time. If a number of exams is greater than a threshold value or the estimated turn-around time for review exceeds a threshold value, then the packaging application may dynamically flag one or more exams to be packaged for third-party readers for quicker review. The packaging application may identify exams for packaging based on routing rules. Additionally or alternatively, the packaging application may identify the exams for packaging based on a number of exam attributes, such as a priority, a patient class, a procedure code, a number of priors, a size of study, and so on. For example, the packaging application may identify an exam with a short patient history for packaging over an exam with a long patient history to decrease the size of the package and also decrease an amount of bandwidth needed to transmit the package. The packaging application may also learn, routines, a number of relevant priors (e.g., patient history details) to package with the exam such that the third-party reader may make an accurate diagnosis and/or report. That is, the packaging application may identify a subset of priors to package with an exam rather than all of the priors. In this way, the packaging application may not only minimize a size of the package and reduce bandwidth needed to transmit the package, but also identify relevant priors for the third-party reader to make the diagnosis and/or the report. The packaging application may compress the package to transmit to the third-party reader and decompress a response package (e.g., results) received from the third-party reader. In this way, the packaging application may automate the steps of identifying relevant priors, packaging the exam and priors, transmitting the package in response to user input, and decompressing a response package received from the third-party reader.

The packaging application may also maintain or interact with (e.g., update) the worklist of exams within the institution. For example, the packaging application may populate a graphical user interface (GUI) with the exams, a status of the exam, a turn-around time (TAT) of the exam, and so on. As such, the user may quickly identify high priority exams and/or overdue exams. Additionally or alternatively, the packaging application may populate a GUI with exams assigned internally (e.g., the institution) and exams transmitted to a target source (e.g., a third-party reader). The packaging application may also monitor new incoming exams and identify if attributes of the new exam match attributes of exams transmitted to the third-party readers. If the attributes match, then the packaging application may flag the exam and/or transmit a notification to the third-party reader to not read the exam. In this way, the packaging application may reduce or eliminate double reads and increase workflow efficiency.

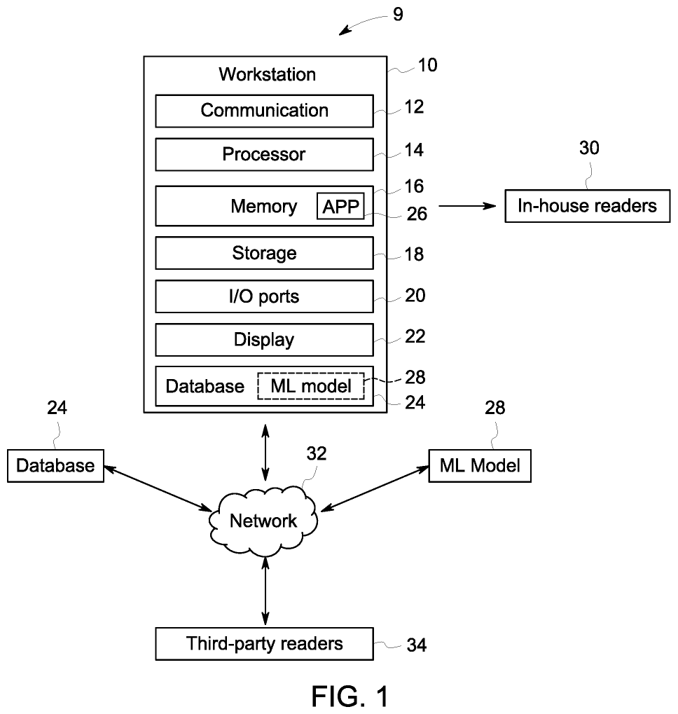

With the preceding in mind, FIG. 1 illustrates an embodiment of work environment 9 including a workstation 10 for acquiring and processing image data, in accordance with aspects of the present disclosure. The work environment 9 may be within an institution, such as hospital, a medical school, a teaching facility, or the like. The workstation 10 may receive raw or pre-processed image data from one or more data sources and process the signals into image data via signal processing techniques, machine-learning routines, artificial intelligence, and so on. The workstation 10 may also receive compressed data from data sources, work schedules, image data (e.g., medical images, exams, cases, digital studies, reports), and the like. For example, a user (e.g., chief radiologist, hospital administrator) may use the workstation 10 to check a worklist of an institution, a status of one or more exams of the worklist, a TAT of the one or more exams, and so on. In certain instances, the workstation 10 may be used to view, comment, annotate, or otherwise manipulate image data (e.g., exams, digital studies, reports, patient history). To this end, the workstation 10 may include any suitable computer device, such as a general-purpose personal computer, a laptop computer, a tablet computer, a mobile computer, and the like that includes specific computer-readable instructions in accordance with present embodiments. In an embodiment, the workstation 10 may include a picture archiving and communications system (PACS) that may store and transmit information captured by medical imaging. For example, the information may include image data such as computed tomography (CT), X-Ray, (positron emission tomography (PET), (single-photon emission computed tomography (SPECT), tomosynthesis, mammography, fluoroscopy, magnetic resonance imaging (MRI), and so on. The PACS may in turn be coupled to a remote client, radiology department information system (RIS), hospital information system (HIS) or to an internal or external network, so that others at different locations may gain access to the raw or processed image data. For example, the PACS may receive a work schedule from the HIS.

The workstation 10 may include various types of components that may assist the workstation 10 in performing various types of tasks and operations. For example, the workstation 10 may include a communication component 12, a processor 14, a memory 16, a storage 18, input/output (I/O) ports 20, a display 22, a database 24, and the like. During operation, the memory 16 may store a packaging application 26 that, when executed by the processor 14, maintains a worklist of exams within the institution, dynamically identifies exams for third-party readers, and packages an exam with associated priors and reports for the third-party readers. To this end, the packaging application 26 may include, access, or be updated using a machine-learning routine that may be trained based on user input over time, the work schedule, the exam attributes, or the like. The database 24 may store a machine-learning model 28 utilized to train the packaging application 26.

The communication component 12 may be a wireless or wired communication component that may facilitate communication between the workstation 10 and various other workstations via a network, the Internet, or the like. For example, the communication component 12 may send or receive image data to or from other workstations 10. In another example, the communication component 12 may send or receive data from the third-party readers 34.

The processor 14 may be any type of computer processor or microprocessor capable of executing computer-executable code. For example, the processor 14 may be configured to receive user input, such as actions performed by the operator, indications to send the package to the third-party reviewer 34, adjustments or readjustments the worklist, identifications of relevant priors and reports, scanning parameters, or the like. The user may select exams for viewing on the workstation 10 or perform one or more other actions. Thus, the operator may select image data for viewing on the workstation 10, perform one or more actions (e.g., identify priors, select exam), verify exams, assign exams, or otherwise operation the workstation 10. Further, the processor 14 may be communicatively coupled to other output devices, which may include standard or special purpose computer monitors associated with the processor 14. One or more workstations 10 may be communicatively coupled for sending or receiving exams, viewing images, storing images, and so forth. In general, displays, printers, workstations, and similar devices supplied with or within the system may be local to the data acquisition components, or maybe remote from these components, such as elsewhere within an institution (e.g., hospital, school), or in an entirely different location, linked to the workstation 10 via one or more configurable networks, such as the Internet, virtual private networks, and so forth. The processor 14 may also include multiple processors that may perform the operations described below.

The memory 16 and the storage 18 may be any suitable articles of manufacture that can serve as media to store processor-executable code, data, or the like. These articles of manufacture may represent computer-readable media (e.g., any suitable form of short-term memory or long-term storage) that may store the processor-executable code used by the processor 14 to perform the presently disclosed techniques. As used herein, applications may include any suitable computer software or program that may be installed onto the workstation 10 and executed by the processor 14. The memory 16 and the storage 18 may represent non-transitory (e.g., physical) computer-readable media (e.g., any suitable form of memory or storage) that may store the processor-executable code used by the processor 14 to perform various techniques described herein. For example, the memory 16 may include machine-learning routines configured to learn the user's preferences for assigning exams for either the in-house readers 30 or the third-party readers 34.

The memory 16 may store the packaging application 26, such as for execution by the processor 14. The packaging application 26, when executed, may identify and maintain a worklist of exams within the institution, predict (e.g., forecast) a need to send exams to third-party readers 34, identify a number of priors and reports to package with the exam, compress the package for the third-party readers 34, and/or transmit the package to the third-party reader 34 in response to user input. As describe herein, the package may include the exam for review, associated priors, reports, or other data needed by the third-party readers 34 to make an accurate diagnosis and/or report. The packaging application 26 may compress the package to reduce a bandwidth needed to transmit the package to the third-party reader 34. The packaging application 26 may also decompress a response file received from the third-party readers 34, thereby streamlining the workflow process within the institution.

The packaging application 26 may also monitor the status of the exams of the worklist as well as a turnaround time of the exams. The packaging application 26 may identify the exams for packaging based on user input and learn (e.g., via machine-learning routines and machine-learning model) the user's preferences over time. As further described herein, the packaging application 26 may also generate a first graphical user interface (GUI) displaying routing rules and a second GUI displaying the worklist of exams within the institution.

The packaging application 26 may be integrated with new and existing workstations 10. For example, the packaging application 26 may be an add-on feature integrated with a software application to streamline a workflow process within the institution. For example, the packaging application 26 may monitor the worklist of exams within the institution, identify the number of exams to be greater than a threshold value, and identify exams for outsourcing to third-party readers 34 to reduce turn-around time and improve quality of service. In this way, the packaging application 26 may improve workflow efficiency within the institution.

Returning to the workstation 10, the I/O ports 20 may be interfaces that may couple to other peripheral components such as input devices (e.g., keyboard, mouse), sensors, input/output (I/O) modules, and the like. The display 22 may operate as a human machine interface (HMI) to depict visualizations associated with software or executable code being processed by the processor 14. In one embodiment, the display 22 may be a touch display capable of receiving inputs from a user of the workstation 10. The display 22 may be any suitable type of display, such as a liquid crystal display (LCD), plasma display, or an organic light emitting diode (OLED) display, for example. Additionally, in one embodiment, the display 22 may be provided in conjunction with a touch-sensitive mechanism (e.g., a touch screen) that may function as part of a control interface for the workstation 10.

Returning to the workstation 10, the database 24 (or multiple databases 24) may store data received by the workstation 10 from one or more data sources. For example, the database 24 may store exams received by the workstation 10, patient history (e.g., priors, reports), image data, digital studies, and the like. The database 24 may also store one or more machine-learning models 28 used by the packaging application 26 to learn user preferences over time, identify exams for the third-party readers 34, and learn relevant priors and reports to package with the exam. The database 24 may also store user preferences over time (e.g., historical user data), which may be used by the packaging application 26 to learn and predict exams for packaging and relevant priors. The database 24 may also store a work schedule of the in-house readers 30 and/or the third-party readers 34. Additionally or alternatively, the database 24 may store one or more service level agreements (SLA) agreements, turn-around time (TAT) of the third-party readers 34, and a quality of the diagnosis or report received from the third-party reader 34, and the like. While the database is illustrated as part of the workstation 10, the database 24 may also be a cloud server or a remote database communicatively coupled to the workstation 10. As illustrated, the database 24 may also be communicatively coupled to the workstation 10.

The database 24 may include or communicate with a machine-learning model 28 that includes historical user data. The application 26 may utilize the machine-learning model 28 to learn to identify exams for packaging and relevant priors associated with the exam. For example, the user may identify one or more relevant priors and reports associated with an exam. In another example, the third-party readers 34 may provide input regarding priors and reports utilized to make a diagnosis and/or the report. Still in another example, the in-house readers 30 may adjust an order of the worklist based on user preferences and/or a priority. The machine-learning model 28 may include the user input over time and be used to train the application 26 to automate the steps of identifying the number of exams being greater than a threshold, identifying exams for packaging, identifying relevant priors and reports associated with the exam, and ordering the exams in the worklist. As illustrated, the machine-learning model 28 may be stored in the database 24 or be communicatively coupled to the workstation 10. For example, the machine-learning model 28 may be located within a remote database or cloud server and communicatively coupled to the workstation 10 via the network 32.

It should be noted that the workstation 10 should not be limited to include the components described above. Instead, the components described above with regard to the workstation 10 are examples, and the workstation 10 may include additional or fewer components relative to the illustrated embodiment. For example, the processor 14 and the memory 16 may be provided collectively within the workstation 10.

In certain embodiments, the workstation 10 may be communicatively coupled to a network 32, which may include collections of workstations, the Internet, an Intranet system, or the like. The network 32 may facilitate communication between the workstation 10 and various other data sources. For example, the network 32 may facilitate communication between a workstation 10 located on the surgery floor and a workstation 10 located in the radiology floor. In another example, the network 32 may facilitate communication between the workstation 10 and the database 24. As described herein, the database 24 may store image data, the machine-learning model 28, patient history (e.g., priors, reports), the worklist, and the like.

The workstation 10 may be accessed by the in-house readers 30 or the third-party readers 34. For example, the workstation 10 may be communicatively coupled to one or more workstations (e.g., mobile computing device, personal laptop, computer, tablet) operable by the in-house readers 30. The workstation 10 may transmit the worklist of exams to the one or more workstations for the in-house reader 30 to review. The in-house readers 30 may be medical professionals, such as radiologists, who are employed by the institution. Each of the in-house readers 30 may have a specialty and may review exams based on a work schedule. For example, the work schedule may include a number of radiologists working over a period of time (e.g., one week, two weeks, one month), a specialty of the radiologists, an experience level of the radiologists, and the like. Based on the worklist and the work schedule, the application 26 may predict a number of exams read over the period of time.

The third-party readers 34 may include medical professionals, such as radiologists, who have a contract or an agreement with the institution, either directly or via a service providing intermediary organization. The third-party readers 34 may include a single medical professional, a group of medical professionals, or an institution that employs medical professionals for reading and reviewing exams. For example, the third-party readers 34 may be telehealth radiologists who are licensed medical professionals. To this end, the institution may implement a service level agreement (SLA) with the third-party reader 34 to outline responsibilities, such as due dates, a quality of the work, a type of work, management of confidential information, and so on. The workstation 10 and/or the institution may be communicatively coupled to the third-party readers 34 via the network 32. As such, the application 26 may package the exam and one or more priors for the third-party readers 34 to read and review. The application 26 may compress the package to decrease a size of the package and reduce a bandwidth needed to transmit the package. The third-party readers 34 may receive the package and perform the review based on the exam and the priors. The third-party reader 34 may make a diagnosis and/or a report based on the contents of the package and transmit the data back to the workstation 10. The application 26 may receive the package and decompress the package for storage within the database 24. As such, the diagnosis and/or report may be easily accessed by the user or the in-house readers 30.

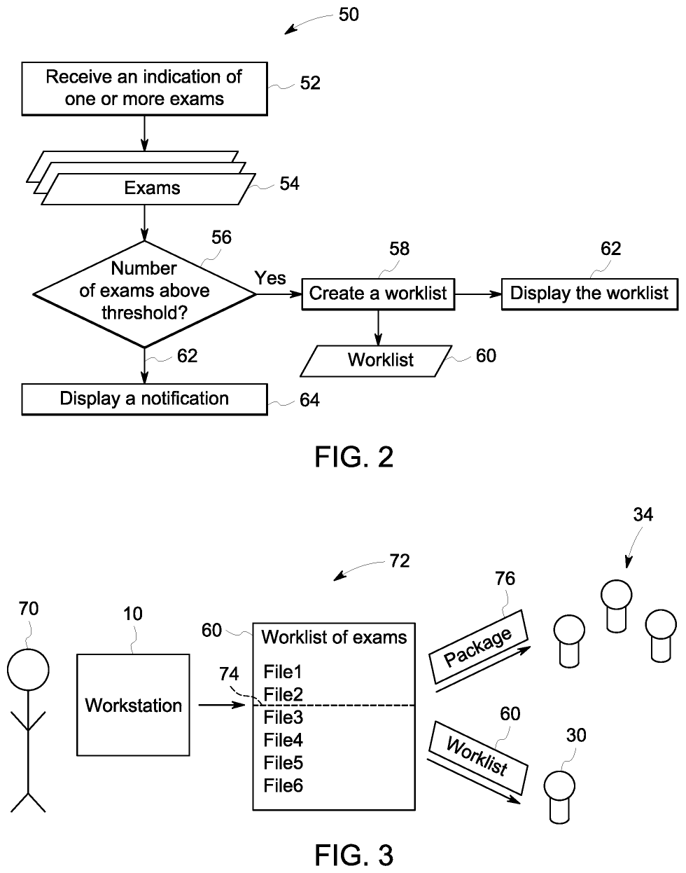

FIG. 2 illustrates a flow chart of an example method 50 to predict (e.g., forecast) a need to package exams for third-party readers 34, in accordance with aspects of the present disclosure. One or more steps of the illustrated method 50 may be performed in a different order from the order depicted in FIG. 2 and/or simultaneously (e.g., in parallel) by the application 26.

At block 52, the application 26 may receive an indication of one or more exams 54 that are ready for review or that are scheduled to be performed. For example, the workstation 10 may receive one or more exams 54 from the data sources and the application 26 may receive an indication of the new exams. In another example, the application 26 may retrieve the exams from the database 24. Still in another example, the user may use the display 22 or I/O ports 20 to input the indication of the exam 54. The user may enter information regarding one or more attributes of the exam, such as a patient name, a patient class, a general physician, a referring physician, an exam date, image data, and the like.

At block 56, the application 26 may determine if the number of exams is above a threshold amount. For example, the application 26 may receive a work schedule for the in-house readers 30 and predict a number of exams that may be reviewed by the in-house readers 30. The work schedule may include a number of radiologists working over a period of time (e.g., one week, two weeks, one month), a specialty of the radiologists, an experience level of the radiologists, and the like. Based on the work schedule, the application 26 may predict a number of exams to be read over the period of time. In this way, the application 26 may predict the threshold amount that corresponds to the number that can or should be reviewed in-house or, correspondingly, a number above which exams should be sent out for review. In another example, the user may input the threshold amount via the display 22. Additionally or alternatively, the application 26 may identify a TAT or a priority of the exams 54 to determine the threshold amount.

If the number of exams is above the threshold amount, then at block 58, the application 26 may create the worklist 60. For example, the application 26 may assign a score to each of the exams 54 and order the exams from a highest score to a lowest score based on their suitability to be sent out or retained for review. The application 26 may also indicate the threshold amount on the worklist 60. In this way, the user may quickly identify the need to outsource one or more exams 54 for third-party reviewers 34.

At block 62, the application 26 may display the worklist 60. For example, the application 26 may populate a GUI with the worklist 60 for display on the display 22 of the workstation 10. The GUI may include the threshold amount, such that the user may quickly determine which exams 54 to assign for in-house readers 30 and which exams 54 to package for third-party readers 30. In another example, the application 26 may store the worklist 60 within the database 24 and the user may access the worklist 60 via the workstation 10.

If the number of exams is below the threshold amount, then at block 64, the application 26 may display a notification. For example, the application 26 may display a pop-up on the display 22 to notify the user that the in-house readers 30 may review the current exams within the institution. In another example, the application 26 may transmit an email, a text message, or an alert to a mobile device of the user.

FIG. 3 illustrates a schematic diagram of the user 70 operating the workstation 10 to assign one or more exams to the third-party readers 34 and the in-house readers 30, in accordance with aspects of the present disclosure. For example, the user 70 may be a chief radiology resident or a hospital administrator. The user 70 may operate the workstation 10 to configure one or more routing rules utilized by the application 26 to create the worklist 60. The workstation 10 may display a graphical user interface (GUI) 72 with the worklist 60. For example, the worklist 60 may include a list with a name or identifier of the exams 54. The application 26 may prioritize the exams 54 from a best exam for the third-party readers 34 to a worst exam for the third-party readers 34, or vice versa. For example, the application 26 may assign a score to each of the exams 54 based on routing rules, attributes of the exam 54, the work schedule of the in-house readers 30, or a combination thereof. As illustrated, FILE1 may be a best exam for the third-party readers 34, followed by FILE2, FILE3, and so on. That is, FILE6 may be the worst exam for the third-party readers 34.

In certain instances, the application 26 may identify a threshold amount of exams 54 the institution may be able to review during a period of time (e.g., one week, two weeks, one month, two months) and indicate the threshold amount on the GUI 72. As illustrated, the threshold amount may be a visual indication on the GUI 72. For example, the line 74 indicates amount number and the exams above the line 74 may be better candidates for third-party readers 34 in comparison to the exams below the line 74. In this way, the user 70 may quickly and efficiently determine which exams to assign to the two groups. Over time, the packaging application 26 may learn the user preferences and/or user input and the predictions may become more granular. In this way, the application 26 may improve the workflow of the institution, such as by automating the evaluation and assignment process.

For example, the user 70 may select the exams above threshold amount for packaging for the third-party readers 34. In another example, the user 70 may select additional exams for packaging to reduce the amount of exams assigned to the in-house readers 30. Based on the user input, the application 26 may adjust the worklist 60 by removing the exams 54 being outsourced to the third-party readers 34. The application 26 may transmit the worklist 60 to the in-house readers 30 for the exams 54 to be reviewed.

Based on the user input, the packaging application 26 creates package 76 to transmit to the third-party readers 34. In certain instances, the application 26 may identify one or more priors and/or reports associated with one exam 54. By way of example, the application 26 may identify the priors and reports associated with FILE3. The application 26 may package FILE3, the priors, and the reports into a single file and compress the file to create the package 76. In this way, the bandwidth needed to transmit the package 76 to the third-party readers 34 may be reduced or minimized. In another example, the application 26 may create the package 76 including all exams 54 (e.g., FILES3-6) to be outsourced to the third-party readers 34. The application 26 may package FILES3-6, the relevant priors and reports, then compress the package 76 for outsourcing. As such, bandwidth needed to transmit the package 76 may be reduced and workflow within the institution may not be affected.

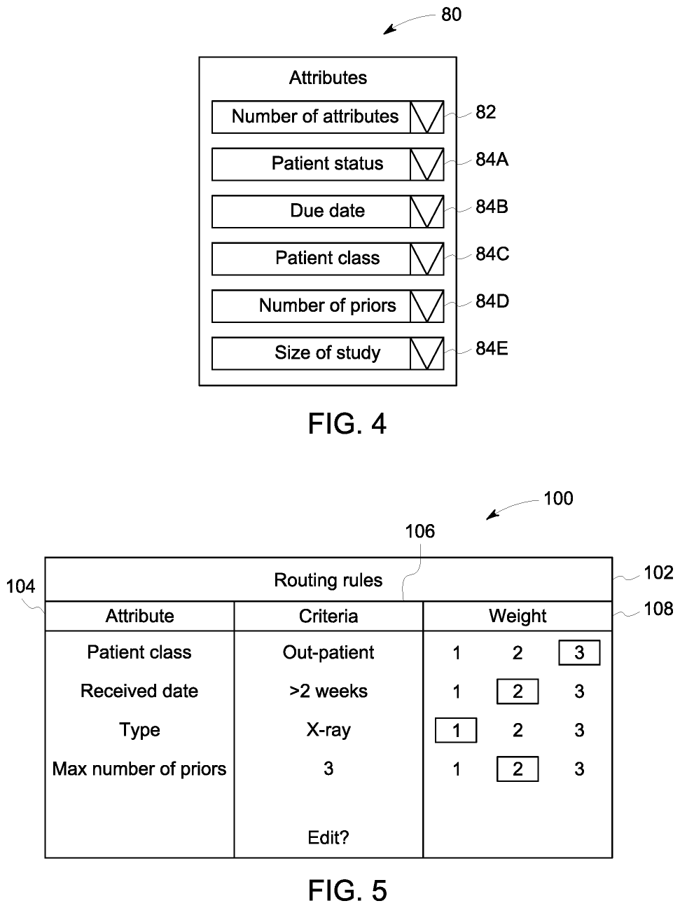

FIG. 4 illustrates a GUI 80 for selecting one or more attributes of the routing rules, in accordance with aspects of the present disclosure. The application 26 may utilize the routing rules to assign a score to the exams 54 and distinguish between exams for the in-house readers 30 and the third-party readers 34. The attributes may be selected by the user and, over time, the application 26 may learn the preferences of the user. The GUI 80 may include an input box 82 and one or more dropdown menus 84. The user 70 may enter a number into the input box 82 to indicate a number of attributes of the routing rules. Based on the number, the application 26 may populate the GUI 80 with one or more dropdown menus 84A-E for the user to select the attributes. The attributes may include a priority (e.g. high, medium, low), a patient class (e.g., in-patient, out-patient), a patient status (e.g., discharged, transferred), a type (e.g., modularity) of exam, a due date of the exam, an exam date, a SLA prioritization (e.g., turn-around time for the type of exam), a size of the exam, a number of priors associated with the exam, and the like. The priority may be determined based on a patient class or a status of the exam. The SLA time may be an amount of time for the third-party readers 34 to review an exam and a TAT time may be an amount of time for the institution to review the exam.

As illustrated, the user 70 may input the number five into the input box 82 and the GUI 80 may be populated with five dropdown menus 84A-E. The user may select one attribute for the routing rules from each of the dropdown menus 84A-E. As illustrated, the user may select “PATIENT STATUS,” “DUE DATE,” “PATIENT CLASS,” “A NUMBER OF PRIORS,” AND “SIZE OF STUDY.” In this way, the user may quickly and easily modify the attributes of the routing rules, thereby modifying the determination made by the application 26.

FIG. 5 illustrates a GUI 100 that displays routing rules 102 used to assign a score to the exams based on the attribute 104, in accordance with aspects of the present disclosure. Based on user input (via the GUI 100), the application 26 may learn the routing rules 102 and identify exams 54 to package for the third-party readers 34. For example, the user 70 may prefer to send out-patient exams to the third-party readers 34 since a priority for out-patient exams may be lower in comparison to a priority for in-patient exams.

As illustrated, the routing rules 102 may include one or more attributes 104, an associated criteria 106, and an associated weight 108. The attributes 104 may be associated with attributes of the exam 54. For example, the attribute 104 may include a patient class, a date the exam was performed, a date the exam was received by the institution, a type (e.g., modularity) of the exam, a procedure code, a number of priors and/or reports, and the like. The application 26 may utilize natural language processing (NLP) or image analysis techniques to identify the attributes of the exam 54. The application 26 may assign a score to the exam 54 based on the routing rules 102 as to whether the exam may be sent to the third-party reader 34 for review.

As described with respect to FIG. 4, the user 70 may select one or more attributes 104 to define the routing rules 102. The user 70 may also define a criteria 106 within the attribute 104 to determine exams 54 for outsourcing. For example, the attribute 104 may be a patient class and the criteria 106 may be out-patient. In other words, the user 70 may prioritize out-patient exams to be sent to third-party readers 34 for review. In another example, the attribute 104 may be the receive date, which may be the date the institution received in the exam. The user 70 may prefer to send older exams to the third-party readers 34, as such the user 70 may set the criteria 106 to greater than 2 weeks. Still in another example, the user 70 may prefer to send certain types of exams to the third-party readers 34. The user 70 may define the criteria 106 as X-ray exams. However, the user 70 may want other types of exams to also be sent, as such the user 70 may assign a lower weight to the attribute 104.

Additionally or alternatively, each attribute 104 may be assigned a weight 108. In certain instances, a higher weight may be associated with a greater preference placed on the attribute 104 in comparison to a lower weight. For example, the user 70 may assign a weight of 3 to patient class, which is higher than the weight of 1 assigned to the type of exam. In this way, the user 70 may indicate that a CT out-patient exam may be prioritized over an X-ray in-patient exam for the third-party readers 34. In another example, the user 70 may prefer to keep exams 54 with a long patient history for the in-house readers 30 to streamline the workflow process. That is, the user 70 may prefer to assign exams 54 with a large number of priors to the in-house readers 30 to reduce the chance of sending a wrong prior to the third-party reader 34 and/or avoid creating a large package that might require high bandwidth to transmit. Additionally or alternatively, the user 70 may prefer large studies (e.g., longitudinal cases) to be reviewed by a same in-house reader 30 to maintain consistency throughout the study. To this end, the user 70 may set a maximum number of priors associated with the exam to 3 and assign a weight of 2. In this way, the application 26 may prioritize exams with less than 3 associated priors for the third-party readers 34. Over time, the application 26 may learn the user's preferences and adjust the routing rules 102. For example, the application 26 may identify certain out-patient exams with three or more priors to package for third-party readers 34 since the weight for patient class is higher (e.g., 3) in comparison to the weight for maximum number of priors (e.g., 2).

While the GUI 100 illustrates routing rules 102 for determining exams for the third-party readers 34, in certain instances the GUI 100 may display routing rules 102 to determine exams for the in-house readers 30. To this end, the user 70 may use a button 110 to edit the routing rules 102. For example, the button 110 may allow the user to edit the attributes 104, the criteria 106, the weight 108, the types of and/or the assignment of exams for the in-house readers 30 or the third-party readers 34, and the like. In this way, the user 70 may quickly and easily assign the routing rules 102 used to determine the exams for the third-party readers 34.

Additionally or alternatively, the application 26 may learn (via machine-learning routines, the machine-learning model 28) to identify exams 54 for packaging based on user preferences. For example, the application 26 may monitor the routing rules 102 over time and identify patterns of user preferences. In another example, the application 26 may adjust the weight applied for an associated attribute based on user input. That is, the user 70 may review the worklist 60 of exams prior to transmitting one or more packages 76 to the third-party readers 34. The user 70 may adjust the worklist 60 and the application 26 may learn the adjustments. In other instances, the user 70 may select certain packages 76 for sending and the application 26 may learn the preferences. Over time, the application 26 may adjust the weight 108 give to each attribute 104. In another example, the user 70 may adjust the attributes 104, the criteria 106, and/or the weight 108 based on the work schedule of the in-house readers 30. For example, the application 26 may identify three neuro readers scheduled for the week based on the in-house reader schedules. As such, the application 26 may prioritize the neuro exams for in-house readers 30. Based on these factors, the predictions of the application 26 may become more granular over time. In this way, the application 26 may streamline the workflow automatically adjusting the routing rules based on machine-learning routines and reduce TAT within the institution.

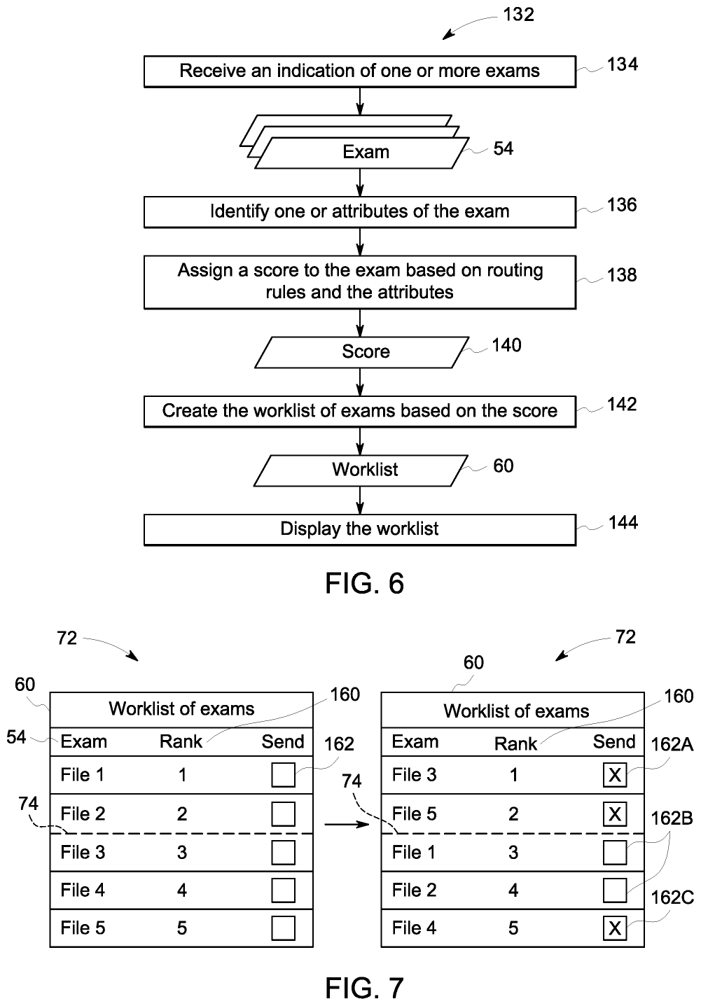

FIG. 6 illustrates a flow chart of an example method 132 for creating the worklist 60 including the exams 54 within the institution based on the routing rules 102, in accordance with aspects of the present disclosure. Based on the routing rules 102, the application 26 may assign a score to each exam 54 within the institution and create the worklist 60 based on the score. The application 26 may also populate a GUI with the worklist 60 and the user 70 may select one or more exams for the third-party readers 34. In this way, the application 26 may predict exams to be packaged, thus streamlining the workflow within the institution, reducing turn-around time, and improving quality of service delivered. One or more steps of the illustrated method 130 may be performed in a different order from the order depicted in FIG. 6 and/or simultaneously (e.g., in parallel) by the application 26.

At block 134, the application 26 may receive indication of one or more exams 54, similar to block 52 described with respect to FIG. 2. For example, the workstation 10 may receive an exam 54 from a data source and the application 26 may receive the indication. At block 136, the application 26 may identify one or more attributes of the exam 54. In certain instances, the application 26 may utilize NLP or image analysis techniques to identify one or more attributes of the exam 54. For example, the application 26 may identify a patient name, a patient class, a referring physician, an exam date, a TAT date, and the like.

At block 138, the application 26 assigns a score to the exam 54 based on the routing rules 102 and the attributes of the exam. For example, the application 26 may compare the attributes of the exam to the routing rules 102 to determine the score. In certain instances, the application 26 may assign a higher score for a greater number of overlapping attributes between the exam 54 and the routing rules 102 in comparison to a lower number of overlapping attributes. The application 26 may adjust the score based on the weight 108 associated with each attribute. For example, the application 26 may analyze a first exam and determine that the type of exam is CT and the patient class is out-patient. The application 26 may compare the type of exam and the patient class to the routing rules 102 to determine the score. In another example, a second exam may be a CT but also part of a longitudinal study. Based on the routing rules 102, the application 26 may automatically assign the second exam a score of zero since the user 70 prioritizes longitudinal studies for the in-house readers. Still in another example, the image data (e.g., exam) may be for a brain CT scan due to a tumor or suspected tumor and a patient may have a number of prior exams related to brain images. The user may compare the current image data to the prior exams (e.g., a longitudinal study) to determine if the tumor is growing, shrinking, changing shape, texture, density, or composition, or remaining the same. As such, the prior exams may be relevant for the review, particularly is assessing the presence, absence, or degree of a growth or change trend. If the packaging application finds a higher number of priors, the packaging application may determine that the patient history is long and/or complex. As such, the packaging application may assign the exam to an in-house reader.

At block 142, the application 26 may create a worklist 60 of the exams 54 based on the score 140. For example, the application 26 may retrieve all the exams 54 within the institution at a period of time. The application 26 may assign a score 140 to each of the exams 54 and create the worklist 60 based on the score. For example, the application 26 may order the exams 54 from a highest score to a lowest score or vice versa to create the worklist 60. At block 144, the application 26 may display the worklist 60, similar to block 62 described with respect to FIG. 2. In this way, the application 26 may automate the process of identifying exams for the third-party readers 34 and the in-house readers 30, thereby improving the workflow within the institution.

FIG. 7 illustrates the GUI 72 displaying the worklist 60 for review prior to transmitting one or more packages 76 to the third-party readers 34, in accordance with aspects of the present disclosure. For example, the application 26 may receive the work schedules of in-house readers 30 to predict a need to package exams for third-party readers 34. The application 26 may automatically identify exams 54 for packaging based on the work schedule, the routing rules 102, machine-learning routines, or a combination thereof. The application 26 may populate the GUI 72 with the worklist 60. For example, the exams 54 may be ordered from a highest score to a lowest score on the worklist 60. The user 70 may (via the GUI 72) adjust the worklist 60 and select one or more exams 54 for packaging. In this way, the user 70 may verify the exams 54 prior transmitting the packages 76 to the third-party readers 34.

Prior to transmitting the packages 76, the application 26 may populate the GUI 72 with the worklist 60. Each exam 54 of the worklist 60 may be associated with a ranking 160. For example, a higher rank (e.g., lower number) may be understood as a best exam for the third-party readers 34 and a lower rank (e.g., higher number) may be a better exam for the in-house readers 30. However, in certain instances, a higher rank may be a best exam for the in-house readers 34 and a lower rank may be a worse exam. The examples above are merely illustrative, and the ranking may be configured to the user preferences.

The ranking 160 may be determined based on the score 140 assigned to each of the exams 54. For example, the worklist 60 may ordered from a highest score to a lowest score or vice versa. As such, the exam with a highest score may be ranked first, followed by the exam with a second highest score, and so on. In this way, the user 70 may quickly identify exams most suited for the third-party readers 34. As illustrated, FILE1 may have a highest score and may be ranked first, FILE2 may have a second highest score and be ranked second, followed by FILE3, FILE4, and FILE 5. That is, FILE1 may have a greater number of overlapping attributes with the routing rules 102 in comparison to FILE5. As such, the application 26 may identify FILE as a better exam to send to the third-party readers 34 and FILE5 as a better exam to assign to the in-house readers 30.

In certain instances, the application 26 may assign a score to each exam based on the routing rules and adjust the score based on machine-learning routines. For example, the user 70 may readjust the ranking of the worklist 60, select one or more exams for the third-party readers 34, or add one or more exams for the in-house readers 30. As illustrated, the user 70 may readjust the worklist 60 by ranking FILE3 first, followed by FILE5, FILE 1. FILE2, and FILE4. For example, the user 70 may drag and drop the exam 54 to adjust the list. In another example, the user 70 may change a ranking of the exam using the ranking column. Then, the application 26 may adjust the worklist and repopulate the GUI 72.

The user 70 may select one or more exams 54 for packaging by selecting a send button 162. As described with respect to FIG. 2, the line 74 may represent the threshold amount of exams the in-house readers 30 may be able to review over a period of time. In certain instances, the user 70 may select one or more exams above the line 74 for packaging. The user 70 may select the send button 162 to indicate to the application 26 that the exam 54 may be transmitted to the third-party readers 34. For example, the user may want to transmit FILE3 to the third-party readers 34. As such the user 70 may select the send button 162A to provide such input. The GUI 72 may include a button allowing the user 70 to select all exams above the line 74 for packaging and the application 26 may automatically package the exams in response the user selecting the button.

The user 70 may not want to package exams below the line 74 since the in-house readers 30 may review the exams in a timely manner. As such, the user 70 may not select the send button 162B (as indicated without the ‘X’) and the application 26 may not transmit the exams, FILE1 and FILE2. As such, the application 26 may automate certain steps of the workflow process and increase efficiency within the institution.

In certain instances, the user 70 may select an exam below the line 74 for the third-party reader 34. As illustrated, the user 70 may want to transmit FILE4 to the third-party readers 43. The user 70 may select the associated send button 162C and the application 26 may package the exam. Such adjustments may be stored in the database 24 as historical user data and the application 26 may learn the adjustments over time. Additionally or alternatively, the application 26 may identify one or more attributes of the exam (e.g., FILE4) and associate the attributes with the adjustments. As such, the application 26 may learn the user preferences over time and adjust the worklist 60 based on the user preferences.

In other instances, the user 70 may not select an exam above the line 74 for the third-party reader 34. That is, the user 70 may repeatedly remove an exam from the exams for transmitting to the third-party readers 34. By way of example, the institution may conduct a long-term PET-CT study, as such, the user 70 may repeatedly remove PET-CT studies from exams being transmitted to the third-party readers 34. The user 70 may assign the PET-CT studies to a certain in-house reader 30 to maintain consistency throughout the study. As such, the application 26 may learn not to include PET-CT studies for third-party readers 34 and learn to assign the PET-CT study to the same in-house reader 30 to maintain consistency of the study. To this end, the application 26 may adjust a score of PET-CT studies to rank the studies at the bottom of the worklist 60 or the application 26 may dynamically flag the PET-CT studies and move them to the bottom of the worklist 60.

In this way, the application 26 may learn (via machine-learning routines) a ranking for the exams based on the user input. As such, the predictions may become more granular and precise over time. Accordingly, the application 26 streamlines the workflow by automatically adjusting the routing rules based on machine-learning routines and reduces TAT within the institution.

Although not illustrated in the GUI 72, the user 70 may also use the GUI 72 to indicate which third-party reader 34 and/or in-house reader 30 the exam may be transmitted to. For example, the third-party readers 34 may include three or more different institutions. As such, the user 70 may indicate that FILE3 may be transmitted to a first third-party reader 34 and FILE5 may be transmitted to a second third-party reader 34. The user 70 may assign the exams to the third-party readers 34 based on a SLA agreement, a TAT time, a quality of the work, and the like. In another example, the user 70 may indicate that FILE1 may be assigned to a first in-house reader 30, FILE2 and FILE4 may be assigned to a second in-house reader 30. Such assignments may also be stored in the database 24 as historical user data and the application 26 may learn the assignments over time. Over time, the application 26 may learn the user preferences and predict the assignments.

While the illustrated example of the GUI 72 includes five exams, in the GUI may display 2, 3, 4, 6, 10, 20, 30, or more exams. For example, the GUI 72 may display 15 a worklist of 15 exams and the user may identify the top ten exams for packaging. In another example, the GUI 72 may display thirty exams and the user may readjust the list, then identify the top five exams for packaging.

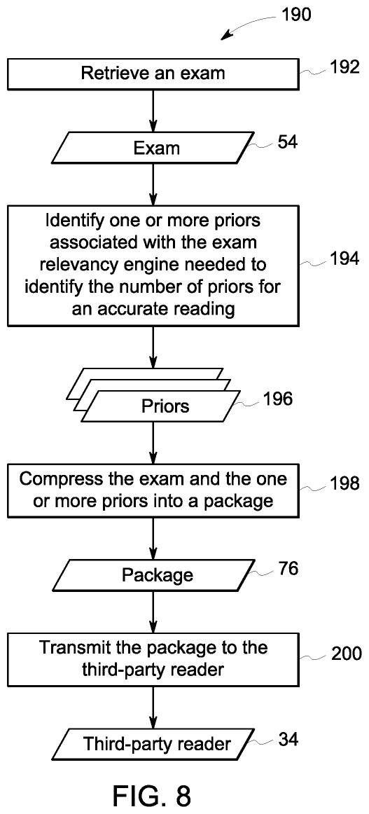

FIG. 8 illustrates a flow chart of an example method 190 to package the exam 54 for the third-party reader 34, in accordance with aspects of the present disclosure. The application 26 may identify the patient history (e.g., priors, reports) associated with the exam 54. It may be beneficial for the application 26 to identify relevant priors and reports to minimize a size of the package 76 and reduce bandwidth needed to transmit the package 76. However, identifying the relevant priors and reports may be crucial for the third-party readers 34 to confidently make an accurate diagnosis with the data in the package 76. As such, the application 26 may learn to determine the priors to package with the exam. One or more steps of the illustrated method 190 may be performed in a different order from the order depicted in FIG. 8 and/or simultaneously (e.g., in parallel) by the application 26.

At block 192, the application 26 may retrieve an exam 54. For example, the user 70 may indicate that the exam 54 may be transmitted to the third-party readers 34 via the GUI 72. The application 26 may retrieve the exam 54 from the database 24. At block 194, the application 26 may identify one or more attributes of the exam 54. As described herein, the application 26 may utilize NPL or image analysis techniques to identify the attributes of the exam. Based on the attributes, the application 26 may identify the patient history. For example, the application 26 may identify a patient name from the exam 54 and identify data within the database 24 based on the patient name.

At block 194, the application 26 may identify one or more priors 196 based on the attributes of the exam 54. For example, the application 26 may determine a number of priors 196 and/or reports needed to make an accurate diagnosis and/or reading. Additionally or alternatively, the application 26 may utilize machine-learning techniques to determine an optimal number of priors 196 needed to make the diagnosis and/or reading. For example, the third-party readers 34 may indicate which priors 196 and/or reports were used to make the diagnosis. In another example, the in-house readers 30 may indicate the priors 196 and reports used during a review. The application 26 may learn from the indications and determine both the optimal number of priors and reports used for review and which priors and reports were used for the review. As such, the application 26 may identify the relevant priors 196 and reports from the patient history.

At block 198, the application 26 may compress the exam 54 and the one or more priors 196 into the package 76. For example, the application 26 may create a folder with the exam 54, the priors 196, and/or the reports. The application 26 may compress the folder to create the package 76. In this way, the application 26 may minimize a size of the package 76 and the bandwidth needed to transmit the package 76.

At block 200, the application 26 may transmit the package 76 to the third-party readers 34. That is, the application 26 may transmit the package 76 via the network 32 to the third-party reader 34. In another example, the application 26 may upload the package 76 to a remote database or a cloud server accessible by the third-party reader 34. As such, the application 26 may streamline the process for the third-party readers 34 to receive all the information needed to review the exam 54.

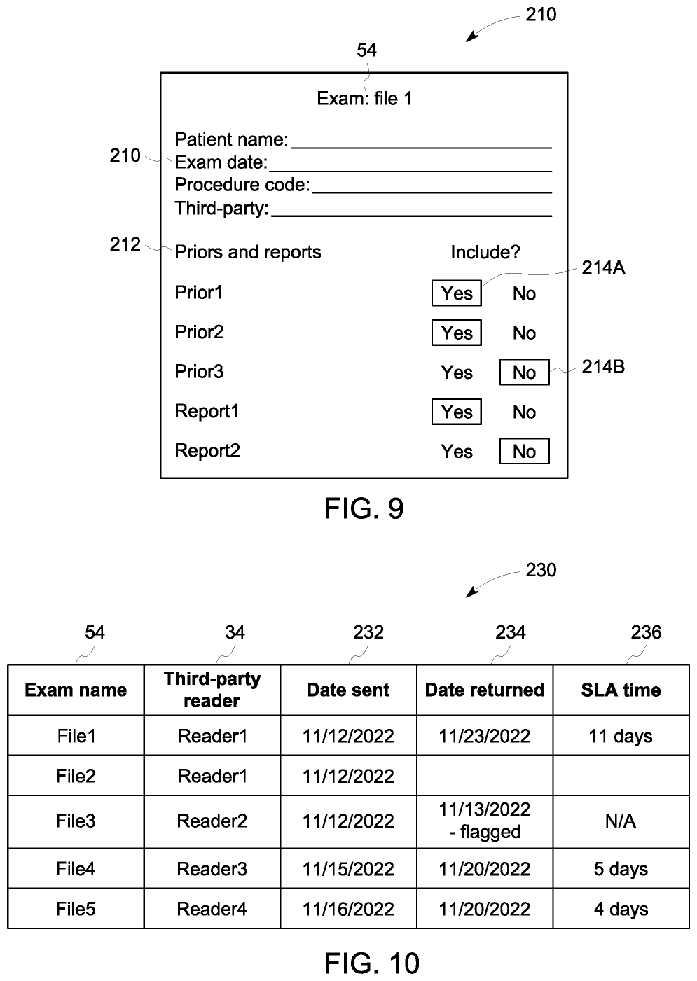

FIG. 9 illustrates a graphical user interface (GUI) 210 displaying aspects of the package 76 prior to transmission, in accordance with aspects of the present disclosure. As described herein, the application 26 may determine a number of priors 196 and reports needed to make an accurate diagnosis and/or report of the exam 54. As such, the package 76 may need to include enough information such that the third-party reader 34 is confident in making an accurate diagnosis. Additionally or alternatively, the application 26 may identify priors and reports that are relevant to the exam 54 being reviewed. In certain instances, the application 26 may populate the GUI 210 with attributes of the exam 54 and the identified priors 196 and reports.

As illustrated, the GUI 210 may include one or more exam attributes 212 and a reference list 214 with the identified priors and reports. The exam attributes 212 may provide information regarding the exam 54 being packaged and sent to the third-party reader 34. For example, the exam attributes 212 may include a patient name, an exam date, a procedure code, a third-party reader. The exam attributes 212 may also include the attributes 104 utilized in the routing rules 102 and/or additional information included in the exam. Based on the exam attributes, the application 26 may identify one or more relevant priors 196 and/or reports to package with the exam 54. The application 26 may populate the GUI 210 with the reference list 214 for user verification. In certain instances, the application 26 may rank the reference list 214 based on a relevancy score assigned to each of the priors 196 and/or reports. For example, a first prior/report may be more relevant to the exam in comparison to a second prior/report. The reference list 214 may be ordered from a most relevant prior/report to a least relevant prior/report. In this way, the application 26 may streamline the review process for the user and improve efficiency.

The user 70 may verify the priors 196 and/or the reports prior to transmission. In certain instances, the user 70 may determine that a prior 196 and/or report may not be relevant to the exam and indicate to the application 26 to remove the prior 196 and/or report from the package 76. The user 70 may use a button 216 to indicate ‘YES’ to include the prior 196 or report or ‘NO’ to not include the prior or report. As illustrated, the user 70 may want to include PRIOR1 and PRIOR2. As such, the user 70 may select the button 216A to indicate to the application 26 that PRIOR1 and PRIOR2 are relevant to the exam. The user 70 may not want to include PRIOR3 and REPORT2 with the exam 54 and/or determine that the documents are not relevant to the exam 54. As such, the user 70 may indicate the selection with the button 216B. The application 26 may receive such inputs and learn to distinguish between relevant and less relevant priors 196 and/or reports. In this way, the application 26 may minimize an amount of patient information transmitted to the third-party reader 34, reduce the size of the package 76, and streamline the workflow process.

In certain instances, the third-party reader 34 may indicate to the application 26 that the number of priors was too much or too little. For example, the third-party reader 34 may indicate which priors 196 and reports were used during the review. In another example, the third-party reader 34 may request additional information to perform the review. As such, the application 26 may adjust the number of priors and reports packaged and/or adjust the type of information included. Over time, the application 26 may learn to identify the priors and reports for packaging. Additionally or alternatively, the application 26 may learn the preferences of the third-party reader 34. For example, a first third-party reader 34 may prefer to have physical exams included with their exams 54 to get a complete picture. In another example, a second third-party reader 34 may prefer to have dose contrast information with the exam 54. Additionally or alternatively, the in-house readers 30 may indicate which priors 196 and/or reports were utilized during a review of the exam 54. Based on the input, the application 26 may learn to automatically identify priors associated with an exam, and more important, relevant priors needed to make an accurate diagnosis. As such, the predictions of the application 26 may be more granular and the size of the package may be decreased. Accordingly, the application 26 may streamline the workflow process.

FIG. 10 illustrates a graphical user interface (GUI) 230 displaying a dashboard with a status of the exam 54 subsequent to transmitting the package 76 to the third-party reader 34, in accordance with aspects of the present disclosure. The application 26 may monitor the outgoing exams 54 for SLA time and TAT time. SLA time may be the amount of time the third-party reviewer 34 took to review the exam and the TAT time may be the time the institution took to deliver the diagnosis or report to a customer (e.g., patient or referring physician). In certain instances, the SLA time may be less than the TAT time. However, in certain instances, the SLA time may be longer than the TAT time. As such, the application 26 may flag the exams that take longer than the allotted or expected time to be reviewed. As such, the user may monitor the exams to make sure that they are coming back in a timely manner.

As discussed herein, the third-party readers 34 and the institution may have an SLA. The SLA may outline a prioritization based on the type of exam, referred to herein as SLA prioritization. For example, an SLA time for a MRI may be 1 week, while an SLA time for a CT may be 2 weeks. In other words, the expected SLA time based on the SLA agreement may change based on the type of image.

As illustrated, the GUI 230 may display the exam 54, an assigned third-party reader 34, a date sent 232, a date returned 234, and a SLA time 236. As described herein, the institution may partner with one or more third-party readers 34 and may assign one or more exams to each of the third-party readers 34. The date sent 232 may be the date the exam 54 was transmitted from the institution to the third-party reader. The date returned 234 may be the date the third-party reader 34 returned a diagnosis or report to the institution. The SLA time 236 may be the amount of time taken by the third-party reader 34 to review the exam.

As illustrated, a first file, FILE1, was transmitted to the third-party reader 34, READER1 on Nov. 12, 2022 and returned Nov. 23, 2022. As such, the SLA time may be 11 days. A second file, FILE2, was transmitted to the third-party reader 34, READER2 on Nov. 12, 2022, but the report may not have been returned to the institution. The application 26 may determine if the turn-around time is greater than the SLA prioritization time, and in certain instances, the application 26 may flag the exam 54 to notify the user 70. As such, the user 70 may monitor the status of outgoing exams 54.

In certain instances, a patient may be an out-patient and a first exam (e.g., FILE3) may be sent to the third-party readers 34 for review and then the patient may become an in-patient. As such, a second exam may be received by the workstation 10 and assigned to the in-house readers 30 (e.g., if out-patient exams are prioritized for third-party readers over in-patient exams). In this case, the in-house reader 30 may review the priors 196 and/or reports associated with the second exam, and a relevant prior may be the first exam. The application 26 may identify this overlap and dynamically flag FILE3 to prevent a double read. The application 26 may pull the first exam from the third-party readers 34 and reassign the first exam to the in-house readers 30. That is, the application 26 may send a notification to the third-party reader 34 to not read the first exam. In this way, the application 26 may streamline the workflow process and also improve efficiency within the institution.

As illustrated, FILE4 and FILE5 may have a diagnosis or report returned. The third-party readers 34 (e.g., READER3, READER4) may transmit or upload a package for the user 70 to retrieve. The package may be a compressed file, a hypertext link, or the like. In certain cases, the application 26 may retrieve the package and decompress the package. As such, the application 26 may translate or transform the package into a readable format by the workstation 10. That is, the application 26 may make it easy to receive a report from the third-party reader 34 using HL7 and/or DICOM standards. In this way, the report may be quickly and easily be entered into the workstation 10.

Technical effects of the invention include automating the steps of identifying that the number of exams is greater than a threshold amount, identifying relevant priors and/or reports, packaging the data, and transmitting the package based on user input. In this way, a size of the package may be minimized, an amount of confidential information within the package may be minimized, and a bandwidth used to transmit the package may be reduced. However, the application may be trained to identify relevant priors and/or reports based on the exam and optimize an amount of data need by the third-party reviews to confidently review the exams. In this way, the application is identifying a subset of data needed to be transmitted with the exam. As such, overall efficiency within the institution and quality of service provided by the institution may increase. Additionally, the application may receive and automatically decompress a package received from a third-party reader.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A system comprising:
a processor-based device storing or accessing a packaging application, wherein the packaging application, when executed by the processor-based device, causes acts to be performed comprising:
determining a number of exams is greater than a threshold number based on a schedule;
assigning a score to each exam of a plurality of exams based on the schedule and on routing rules;
creating a worklist comprising the plurality of exams, wherein the plurality of exams is ordered in the worklist from a highest score to a lowest score;
packaging an exam of the plurality of exams with additional data based on user input; and
transmitting the package to a third-party reader.

2. The system of claim 1, wherein the packaging application, when executed, is configured to:
decompress a file from the third-party reader, wherein the file comprises a report or a diagnosis; and
associate the report or the diagnosis with the exam.

3. The system of claim 1, wherein the packaging application, when executed, is configured to:
generate a graphical user interface (GUI) displaying the worklist; and
receive user input selecting the exam from the plurality of exams for packaging.

4. The system of claim 3, wherein the packaging application, when executed, is configured to package the exam of the plurality of exams with additional data by:
identifying a patient history associated with the exam, wherein the patient history comprises a plurality of priors; and
determining one or more relevant priors of the plurality of priors based on one or more attributes of the exam.

5. The system of claim 4, wherein the packaging application, when executed, is configured to:
generate a second GUI displaying the one or more relevant priors; and
adjusting the one or more relevant priors based on user input.

6. The system of claim 1, wherein the packaging application, when executed, is configured to:
generate a GUI comprising the routing rules, wherein the routing rules comprise a priority, a patient class, a type of exam, and a maximum number of priors; and
assign a weight to each of the routing rules based on user input.

7. The system of claim 1, wherein the packaging application, when executed, is configured to:
maintain an outgoing list of one or more transmitted exams transferred to the third-party reader; and
generate a GUI displaying the outgoing list, a service level agreement time, and a turn-around time.

8. The system of claim 7, wherein the packaging application, when executed, is configured to:
determine a received exam matches a transmitted exam of the one or more transmitted exams of the outgoing list;
flag the exam on the outgoing list;
generate a notification on the GUI in response to flagging the exam;
transmit an additional notification to the third-party reader to stop reviewing the exam; and
assign the exam to an in-house reader.

9. The system of claim 1, wherein the schedule comprises a number of in-house readers working over a period of time and a specialty of each of the in-house readers, and wherein the packaging application, when executed, is configured to predict the threshold number the in-house readers may review in the period of time.

10. The system of claim 1, wherein the packaging application, when executed, is configured to:
identify the exam of the plurality of exams as part of a long-term study; and
reassign the exam for an in-house reader based on the identification.

11. A method comprising:
determining, via a processor, that a number of a plurality of exams is greater than a threshold number based on a schedule of in-house readers;
assigning, via the processor, a score to each of the plurality of exams based on routing rules;
creating, via the processor, a worklist comprising the plurality of exams, wherein the plurality of exams is ordered based on the score;
generating, via the processor, a graphical user interface (GUI) comprising the worklist; and
transmitting, via the processor, an exam of the plurality of exams to a third-party reader in response to user input.

12. The method of claim 11, comprising:
identifying, via the processor, one or more priors associated with the exam; and
determining, via the processor, a relevant prior of the one or more priors to transmit with the exam.

13. The method of claim 12, wherein determining, via the processor, the relevant prior comprises:
assigning, via the processor, a score to the one or more priors based on one or more attributes of the exam;
creating, via the processor, a list of the one or more priors based on the score; and
receiving, via the processor, user input identifying a relevant prior from the list.

14. The method of claim 13, comprising:
compressing, via the processor, the relevant prior and the exam into a package;
wherein transmitting, via the processor, comprises transmitting the package to the third-party reader in response to user input.

15. The method of claim 11, comprising:
generating, via the processor, the GUI comprising the worklist and the threshold number; and
adjusting, via the processor, the worklist in response to user input.

16. The method of claim 11, comprising:
generating, via the processor, an additional graphical user interface displaying the routing rules; and
receiving, via the processor, a preference and a weight for each of the routing rules.

17. A non-transitory, computer-readable medium comprising computer-readable code, that when executed by one or more processors, causes the one or more processors to perform operations comprising:
determining that a number of a plurality of exams is greater than a threshold number based on a schedule of in-house readers;
assigning a score to each of the plurality of exams based on routing rules;
creating a worklist comprising the plurality of exams, wherein the plurality of exams is ordered based on their respective scores;
generating a first graphical user interface (GUI) comprising the worklist and the threshold number;
receiving a user input selecting an exam from the worklist to transmit to a third-party reader;
identifying one or more priors based on attributes of the exam;
compressing the exam and the one or more priors into a package; and
transmitting the package to the third-party reader.

18. The non-transitory, computer-readable medium of claim 17, wherein the computer-readable code, when executed by the one or more processors, causes the one or more processors to perform further operations comprising:
generating a second GUI comprising the routing rules, wherein the routing rules comprise an attribute, a criteria, and a weight; and
adjusting the routing rules based on user input.

19. The non-transitory, computer-readable medium of claim 17, wherein the computer-readable code, when executed by the one or more processors, causes the one or more processors to perform further operations comprising;
receiving user input assigning the exam from the worklist to an in-house reader; and
adjusting the routing rules based on the user input.

20. The non-transitory, computer-readable medium of claim 17, wherein determining the number of the plurality of exams comprises:
identifying a number of in-house readers and a schedule of the in-house readers based on the schedule;
estimating the number of exams the in-house readers can review over a period of time, wherein the estimation is the threshold number; and
comparing the estimation to the number of the plurality of exams.
